(19) United States
(12) Patent Application Publication
(10) Pub. No.: US 2005/0131649 A1
(43) Pub. Date: Jun. 16, 2005

(54) Advanced databasing system for chemical, molecular and cellular biology

(76) Inventors: Christopher N. Larsen, Astoria, NY (US); Brian Osborne, Nyack, NY (US); Chuyu Ren, Jersey City, NJ (US); Lin Bai, Plainsboro, NJ (US); Grace Stafford, Irvington, NY (US); Dmitri Evstratovski, New York, NY (US); David Rubin, Maplewood, NJ (US)

Correspondence Address: MORGAN & FINNEGAN, L.L.P., 3 World Financial Center, New York, NY, 10281-2101, US

(21) Appl. No.: 10/916,842

(22) Filed: Aug. 12, 2004

Related U.S. Application Data
(60) Provisional application No. 60/494,364, filed on Aug. 12, 2003.

(51) Int. Cl. G06F 19/00; G01N 33/48; G01N 33/50

(52) U.S. Cl. 702/19; 702/20

(57) ABSTRACT

The present invention relates to systems and methods for biomedical drug research, addressing major molecular, cell biological and biochemical information management issues within drug discovery and basic biomedical science. The invention allows scientists to enter biological, chemical, and/or molecular data into a central database, analyze the data entries according to entry attributes, and graphically view the results. A group of web-enabled researchers can enter, share and analyze molecular and cellular data and information from the resources using standardized vocabularies and ontologies. This application describes in detail components of the databasing system, including but not limited to annotation modules, reference managers, advanced search algorithms, ontology browsers, molecular network builders, and text processing scripts. Ultimately, the information gathered, viewed, and analyzed by this relational databasing system is relevant to research ranging from basic researchers to advanced research in applied technologies within pharmaceutical development and biotech fields.

This application claims the benefit of U.S. Provisional Application No. 60/494,364 filed Aug. 12, 2003 which is herein incorporated by reference.

### FIELD OF THE INVENTION

This invention relates to the field of biomedical information management and research. More specifically, the invention relates to a system and method for extracting public and private chemical, biological and/or molecular research data, integrating the extracted information with entries in a relational database, and providing utilities for analyzing the stored data and information.

### BACKGROUND INFORMATION

Generally, lifescience researchers need an information management system that allows its users to easily comprehend the extremely complex and vast amounts of data associated with chemical, biological, and/or molecular research. There has not been a comprehensive research system and method including 1) relational database schema and tables, 2) standardized ontologies and vocabularies, 3) scientific applications, and 4) user friendly data annotation services. Previous attempts at similar systems gave rise to individual modules and do not provide the full range of capabilities and functionality of the present invention. For example, past attempts to annotate data in a database often required a human curator to manually input data as free text “comments.” The data appended with stored data entries in such systems is collected and saved in non-standardized data formats, impeding interaction with other data processing systems.

Previous attempts at research information management systems have developed in narrow applications. One such system references sequence data related to proteins, genes and gene loci. Generally, such sequence data related to proteins, genes and gene loci are archived in publicly available databases. Such systems may append a sequence citation to a data entry. Such systems simply append source reference citations as internet hyperlinks and do not require a literature source reference citation. Other systems allow the capture of data regarding a single type of molecular data or protein functions, but do not include the source references for the data. There is a system that defines a hierarchical structure for annotating protein functions. However, the system does not integrate information about tissue specificity, cellular processes, sub-cellular localization, disease associations, mutations, modifications, molecular genetics, molecular complexes, compound registries, interaction networks, and gene linkages, as does this invention.

Previous attempts of displaying processed data include a system that displays processed data related to interactions associated with a specific yeast protein. Another system displays analyzed data related to simple interactions for a virus in a yes/no format. However, neither system interacts well with a relational database. Yet another system for analyzing molecular interactions displays signal transduction interaction networks as either text-based cascades or flatly drawn maps. The system does not draw molecular networks dynamically, and furthermore the system does not readily interact with the database used to store the displayed data.

A few other commercial applications have been created for viewing protein interaction maps, but such applications do not address biochemical intermediates, or drug compounds. These applications do not provide additional information related to the actual interaction, such as the type of interaction (Binding, covalent modification, etc . . . ), the interaction logic (activates, inhibits), the effects (cleavage, phosphorylation), or the downstream ramifications to the cellular process (cellular growth, cell division). Generally, previous attempts at analyzing and displaying interactions simply show data content and do not facilitate data processing, analysis or new data entry.

Generally, the prior process involving an individual user manually appending non-standardized data to data entries in a molecular life science database has not been a scalable, extensible, or efficient method for collecting, analyzing, or archiving data related to chemical, biological, and/or molecular data entries. Further, such practices seriously impede a user attempting to research aspects of complex interactions between data entries.

### SUMMARY OF THE INVENTION

The invention provides a comprehensive method and system allowing a researcher to break down the often complex and overwhelming amount of data related research findings into manageable easy-to-understand visual representations. Generally, the system focuses on two facets of scientific research: data annotation and data analysis. The annotation module uses ontology browsers, pull down menus filled with standard search terms, and reference managers, which populate the information management system with relevant data. The analysis module employs advanced search algorithms, network builders, and tools to examine data already present in the system. Unencumbered access to the interactions of chemical, biological, and/or molecular data is critical for advances to occur in related scientific research. Without a central system for organizing, entering, and analyzing data, experiments may be repeated, performed without knowledge of prior findings and inappropriately prioritized. The present invention creates a method, system and apparatus for resolving the shortcomings of the systems discussed above. Hereafter, the system may be referred to as Cognia Molecular™ or CM™.

Other and further aspects of the invention will become apparent from the following detailed description with reference to the accompanying drawings.

### BRIEF DESCRIPTION OF THE DRAWINGS

FIG 1A. is an exemplary overview of a three-tier architecture system associated with the Cognia Molecular™ system.

FIG. 1B is an exemplary overview of the Cognia Molecular™ system

FIG. 2 is an exemplary screen from Cognia Molecular™ system's logon entry screen.

FIG. 3 is an exemplary screen of an embodiment of an annotation module and a preliminary search page.

FIG. 4 is an exemplary screen of a universal ontology hierarchy browser

FIG. 5 is an exemplary screen of search results associated with an ontology browser's search engine.

FIG. 6 is an exemplary screen associated with the process of adding attribute terms to the annotation in the ontology browser.

FIG. 7 is an exemplary screen of a reference manager entry point.

FIG. 8 is an exemplary screen of the launched reference annotation module.

FIG. 9 illustrates the exemplary results of a search based on PubMed identification numbers.

FIG. 10 is an exemplary screen shot associated with the referencing module saved results.

FIG. 11 is an exemplary generic reference addition to the database.

FIG. 12 is an exemplary screen shot of a network builder entry point.

FIG. 13 is an exemplary illustration created by a molecular interaction builder.

FIG. 14 is an exemplary screen shot that illustrates functionality associated with molecular interaction builder.

FIG. 15 illustrates an exemplary filter apparatus for eliminating extraneous interactions in a drawn network.

FIG. 16 is an exemplary screen shot of an advanced search screen.

FIG. 17 is an exemplary screen shot of an advanced search screen implementing a Boolean recombined search.

FIG. 18 is an exemplary screen shot of a user-specific saved advanced search.

FIG. 19 is an exemplary screen shot displaying results obtained during an advanced search.

FIG. 20 is an exemplary screen shot of an output table corresponding to a data entry from the Cognia Molecular™ database system.

FIG. 21 is an exemplary screen shot of a representative user interface associated with an interaction loader.

FIG. 22 is an exemplary screen shot of a user interface for curation menus associated with the Cognia Molecular™ database system.

### DETAILED DESCRIPTION OF THE INVENTION

In the following description of the various embodiments of the invention, reference is made to the accompanying drawings, which form a part hereof, and in which is shown by way of illustration various embodiments in which the invention may be practiced. It is to be understood that other advancing embodiments may be utilized, and structural and functional modifications may be made, without departing from the scope of the present invention.

Overview

This invention provides a comprehensive method and system for storing, searching, and analyzing the vast amount of knowledge present in published and private scientific literature. Without databases that aggregate and reference facts about cellular and molecular biology, research efforts in drug discovery and basic research are significantly hampered by tangled or unconnected data and research information.

The system is a unified solution of an organized IT system used for pharmaceutical research. As such, it is not simply a database per se, nor is it simply a body of database content. Rather, it is a method and system for capturing, downloading, annotating, analyzing, and sharing data about the molecular sciences. Cognia Molecular™ is an integrated platform for data entry, sharing, and analysis based on researching component modules in coordination with a relational database, its tables, the user interface, accepted ontologies, and visualization tools. The system is an expansive tool enabling researchers to centralize and analyze complex data associated with interactions between chemical, biological, and/or molecular data entries, among other things.

Described herein are various aspects of the Cognia Molecular™ research information management system. One such aspect of the present invention is a network builder that constructs interaction networks between molecules in the context of other cellular proteins. The molecular interaction networking tool, when coupled with an underlying relational database, forms a graphical output of any tabulation or collection of interaction data regarding biomolecules. The network builder module implements a database driven, force directed node-edge display format for displaying database interaction content. One of the advantages of the Cognia Molecular™ network builder involves coupling the system to an underlying robust relational database. Scientific research functionality is bolstered by features of the Cognia Molecular™ system. For example, a user may create interaction maps that are multi-layered that allow a user to easily access the underlying data used to create the representative molecular interaction.

Another aspect of the present invention involves an advanced search capability of the Cognia Molecular™ system, which allows for the detailed search and retrieval of biomolecular information. Users of the Cognia Molecular™ system can search and retrieve information regarding biomolecules and bioactive compounds form a variety of resources. The advanced search capability described herein can locate proteins based a wide range of search terms including, but not limited to: their molecular weight, sequence length, structural motifs, sequence domains, names, synonyms, tissue specificity, cell cycle expression, and presence during development and differentiation. By way of example only, a synonym may be a secondary database identifier such as “UCHBL1”, or a name referring to the identical molecule as reported in the literature such as “UCH-Low mass number 1.”

The characteristics provide useful tags whereby the vast amount of biomedical information present in databases can be queried. Recombination of searches in a Boolean fashion allows the searches to be even more useful, as several characteristics of proteins can be recombined into a single set of search terms, enabling a user to create an extremely focused search. This leads to eliminating extraneous search results and providing more detailed searches, more relevant results, and greater speed of use and utility for the users. Previous databases and systems were not able to perform these searches because they either did not capture such types of information or were unable to create detailed searches based on these molecular characteristics.

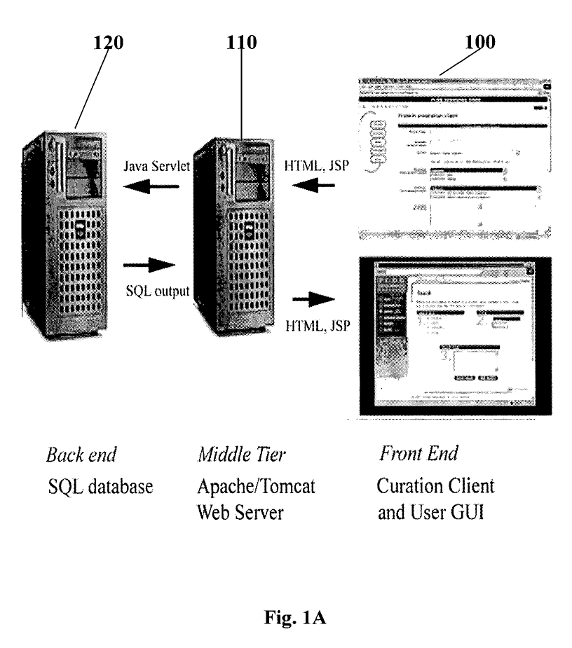

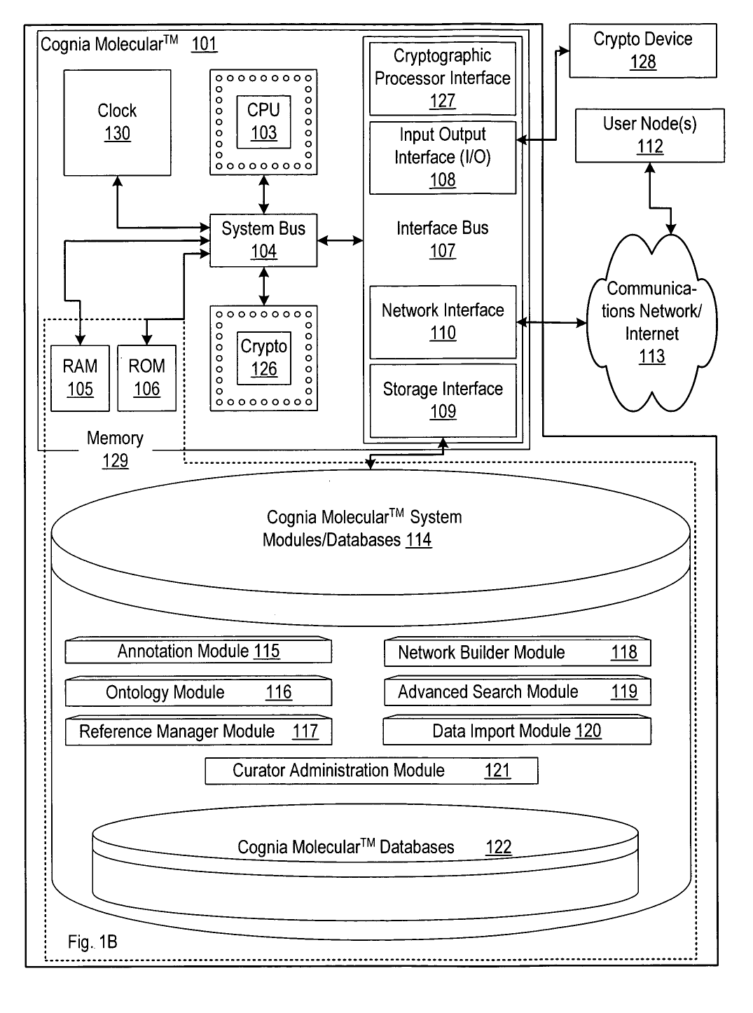

Described herein are the features of the various modules of the Cognia Molecular™ databasing system, such as annotation modules, reference managers, advanced search algorithms, ontology browsers, molecular network builders, applets, and text processing scripts. The three-level architecture system illustrated in FIG. 1A, includes a user's web application node 100, running the front end graphical user interface, implementing the annotation module of the CM™ system, as well as the other modules described in detail below. It is to be understood that depending on the actual implementation, the various modules may be situated on different hosts. The user node communicates with a middle-tier web server 110, acting as the intermediary between the back end relational database 120, the user node 100, and the internet (not shown).

FIG. 1A illustrates the server side functionality for an embodiment of the invention. It is to be understood that the various operational modules, database modules and hardware elements may be supplemented to achieve additional functionality and that the embodiment illustrated herein is non-limiting.

FIG 1B is an exemplary diagram illustrating system elements associated with an embodiment of Cognia Molecular™ (CM™). Cognia Molecular™ 101 may be connected to and/or communicate with entities such as, but not limited to: one or more user nodes 112 connected through a communications network and/or the internet 113. Depending on the actual implementation, the system may even be connected to and/or communicate with a cryptographic processor device 128.

The Cognia Molecular™ system 101 may comprise a clock 130, central processing unit (CPU) 103, a read only memory (ROM 106), a random access memory (RAM 105), and/or an interface bus 107, and conventionally, although not necessarily, are all interconnected and/or communicating through a system bus 104. The system clock typically has a crystal oscillator and provides a base signal. The clock is typically coupled to the system bus and various means that will increase or decrease the base operating frequency for other components interconnected in the computer systemization. The clock and various components in a computer systemization drive signals embodying information throughout the system. Such transmission and reception of signals embodying information throughout a computer systemization may be commonly referred to as communications. These communicative signals may further be transmitted, received, and the cause of return and/or reply signal communications beyond the instant computer systemization to: communications networks, input devices, other computer systemizations, peripheral devices, and/or the like. Optionally, a cryptographic processor 126 may similarly be connected to the system bus. Of course, any of the above components may be connected directly to one another, connected to the CPU, and/or organized in numerous variations employed as exemplified by various computer systems.

The CPU 103 comprises at least one high-speed data processor adequate to execute program modules for executing user and/or system-generated requests. The CPU 103 may be a microprocessor such as the Intel Pentium Processor and/or the like. The CPU 103 interacts with memory through signal passing through conductive conduits to execute stored program code according to conventional data processing techniques. Such signal passing facilitates communication within the Cognia Molecular™ and beyond through various interfaces.

Interface Adapters

Interface bus(es) 107 may accept, connect, and/or communicate to a number of interface adapters, conventionally although not necessarily in the form of adapter cards, such as but not limited to: input output interfaces (I/O) 108, storage interfaces 109, network interfaces 110, and/or the like. Optionally, cryptographic processor interfaces 127 similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (PCI), Personal Computer Memory Card International Association (PCMCIA), and/or the like.

Storage interfaces 109 may accept, communicate, and/or connect to a number of storage devices such as, but not limited to: storage devices comprising system modules/databases 114, removable disc devices, and/or the like. Storage interfaces may employ connection protocols such as, but not limited to: (Ultra) Advanced Technology Attachment (Packet Interface) ((Ultra) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces 110 may accept, communicate, and/or connect Cognia Molecular™ with a communications network/the internet 113 and in turn, with user node(s) 112. Network interfaces may employ connection protocols such as, but not limited to: direct connect, Ethernet (thick, thin, twisted pair 10/100/1000 Base T, and/or the like), Token Ring, wireless connection such as IEEE 802.11 a/b/g, Bluetooth, and/or the like. A communications network may be any one and/or the combination of the following: a direct interconnection; the Internet; a Local Area Network (LAN); Storage Area Network (SAN), Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface.

Input Output interfaces (I/O) 108 may accept, communicate, and/or connect to cryptographic processor devices 128, alternate system input device (not shown) and/or the like. I/O may employ connection protocols such as, but not limited to: Apple Desktop Bus (ADB); Apple Desktop Connector (ADC); audio: analog, digital, monaural, RCA, stereo, and/or the like; IEEE 1394; infrared; joystick; keyboard; midi; optical; PC AT; PS/2; parallel; radio; serial; USB; video interface: BNC, composite, digital, RCA, S-Video, VGA, and/or the like; wireless; and/or the like. A common output device is a video display, which typically comprises a CRT or LCD based monitor with an interface (e.g., VGA circuitry and cable) that accepts signals from a video interface. The video interface composites information generated by a computer systemization and generates video signals based on the composited information. Typically, the video interface provides the composited video information through a video connection interface that accepts a video display interface (e.g., a VGA connector accepting a VGA display cable).

User node device(s) 112 may be connected and/or communicate with or to I/O Interface 108 and/or with or to other facilities of the like such as network interfaces 110, storage interfaces 109, and/or the like. A user node device 112 may be connected with a range of peripheral devices configured to interact with a user. Such peripherals may include cameras, dongles (for copy protection, ensuring secure transactions as a digital signature, and/or the like), external processors (for added functionality), goggles, microphones, microscopes, anatomical or cellular imaging systems, monitors, network interfaces, printers, scanners, storage devices, visors, and/or the like.

Cryptographic units such as, but not limited to, microcontrollers, processors 126, interfaces 127, and/or devices 128 may be attached, and/or communicate with Cognia Molecular™. A MC68HC16 microcontroller, commonly manufactured by Motorola Inc., may be used for and/or within cryptographic units. Equivalent microcontrollers and/or processors may also be used. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Other commercially available specialized cryptographic processors include VLSI Technology's 33 MHz 6868, Mykotronx 24 MHz MYK-82A, or Semaphore Communications' 40 MHz Roadrunner 284.

Memory

A storage device for storing the system modules/databases 114 may be any conventional computer system storage. Storage devices may be a fixed hard disk drive, and/or other devices of the like. However, it is to be understood that Cognia Molecular™ may employ various forms of memory 129 and that the various modules comprising the system are not limited to residing in the same memory. In a typical configuration, memory 129 will include ROM, RAM, and a storage device 114. Generally, any mechanization and/or embodiment allowing a processor to affect the storage and/or retrieval of information is regarded as memory 129. Thus, a computer systemization generally requires and makes use of memory. However, memory is a fungible technology and resource, thus, any number of memory embodiments may be employed in lieu of or in concert with one another.

Module Collection

The storage device 114 may contain a collection of program and/or database modules and/or data such as, but not limited to: an annotation module 115; ontology module 116; a reference manager module 117; a network builder module 118; an advanced search module 119; data import module 120; a curator administration module 121; and Cognia Molecular™ databases 122. These modules may be stored and accessed from the storage devices and/or from storage devices accessible through an interface bus. Although non-conventional software modules such as those in the module collection, typically and are stored in a local storage device 114, they may also be loaded and/or stored in memory such as: peripheral devices, RAM, remote storage facilities through a communications network, ROM, various forms of memory, and/or the like. The functionality associated with the Cognia Molecular™ modules and databases will be described in greater detail below.

The Cognia Molecular™ database 122 may be embodied in a database that is stored program code and executed by the CPU. The stored program code portion configures the CPU to process the data stored in the database. The databases may be conventional, fault tolerant, relational, scalable, extensible and secure databases. Relational databases are an extension of a flat file, and are collections of such. Specifically, relational databases such as used by this invention consist of a series of related tables. The tables are interconnected via a key field and/or table constraints. Use of the key field allows the joining or combination of the tables by indexing against the key field; i.e., the key fields act as dimensional pivot points for combining or selecting information from various tables. Relationships generally identify links maintained between tables by matching primary or logical keys. Primary or logical keys represent fields that uniquely identify the rows of a table in a relational database. More precisely, they may uniquely identify rows of a table on the “one” side of a one-to-many relationship, or one-to-one relationship. Because of the breadth of knowledge able to be imported into the present embodiment, this invention heavily utilizes a flexible, non-redundant key system for its unique and powerful abilities.

Alternatively, the Cognia Molecular™ databases may be implemented using various standard data-structures, such as an array, hash, (linked) list, struct, table, and/or the like. Such data-structures may be stored in memory and/or in (structured) files. If the Cognia Molecular™ databases are implemented as data-structures, the use of the Cognia Molecular™ databases may be integrated into another module such a data management module. Databases may be consolidated and/or distributed in countless variations through standard data processing techniques. Portions of databases, e.g., tables, may be exported and/or imported and thus decentralized and/or integrated.

In an alternative embodiment, such tables are capable of being decentralized into their own databases and their respective database controllers (i.e., individual database controllers for each table). Of course, employing standard data processing techniques, one may further distribute the databases over several computer systemizations and/or storage devices. Similarly, configurations of the decentralized database controllers may be varied by consolidating and/or distributing various database modules.

Cognia Molecular™ databases may communicate to and/or with other modules in a module collection, including themselves, and/or facilities of the like. The databases may contain, retain, and provide information regarding other user nodes and data.

Finally, it is to be understood that the logical and/or topological structure of any combination of the module collection and/or the present invention as described in the figures and throughout are not limited to a fixed execution order and/or arrangement, but rather, any disclosed order is exemplary and all functional equivalents, regardless of order, are contemplated by the disclosure. Furthermore, it is to be understood that such structures are not limited to serial execution, but rather, any number of threads, processes, services, servers, and/or the like that may execute asynchronously, simultaneously, synchronously, and/or the like are contemplated by the disclosure.

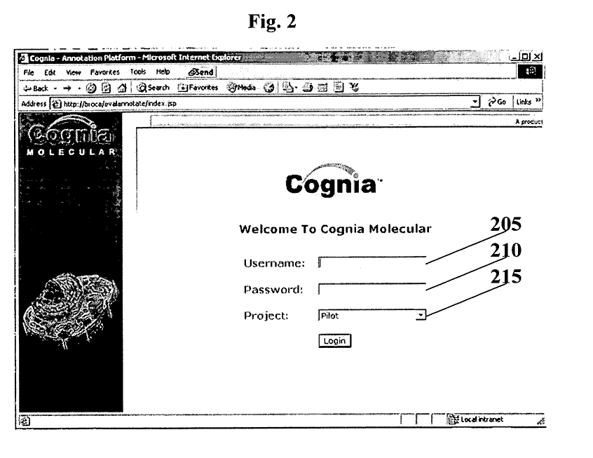

FIG. 2 illustrates an exemplary user entry screen implemented on the user node 100. Depending on the actual implementation, a user may be requested to provide a username 205 and a password 210. In the embodiment shown in FIG. 2, the user is required to provide a specific project 215. The system may provide limited access to certain users based on either the type of project entered or on the specific project 215.

In one embodiment of the present invention, the software modules of the system implemented to accomplish the functionality described herein were created using HTML, dynamic HTML, XML, C, Java 2, SQL, PL/SQL, Perl, Javascript, Ruby, Python, visual basic, and Java Server Pages (JSP) software code. However, it is to be understood that the modules may be created by using any of a range of computer programming languages, and/or scripting languages. Further, it is to be understood that the software modules and the corresponding hardware for implementing the invention vary based on actual the application. In one embodiment, the software modules are implemented on a computer system with a 32 or 64-bit operating system, a web browser such as Microsoft Internet Explorer 5 or later, and a database such as a three-tiered Oracle relational database architecture that is J2EE compliant and web-capable. Although alternative embodiments may be implemented, this embodiment also includes either a local installation of, or a network connection connected to, a databasing system.

Annotation Module

Data entry into the system is achieved by the use of a module including annotation pages. One possible embodiment of such annotation pages involves HTML/Java based software utilities for capturing data, information, and/or characteristics related to a specific research source. A user may select a chemical, biological, or molecular data entry from a wide range of public and private biomedical data resources. The Cognia Molecular™ system includes modular components, which together define a databasing system used in basic research and industrial drug discovery, in an effort to capture, process, and analyze the large amount of knowledge present in published and private scientific resources.

In an embodiment of the present invention implementing web browser technology, the annotation module uses over 2000 pre-defined terms to describe the biology and chemistry of applied pharmaceutical and biotech research, as well as basic scientific research. Software scripts streamline the process of data entry, and various technologies facilitate standardizing the data entry, assure quality control of the data, and increase the accuracy of the input.

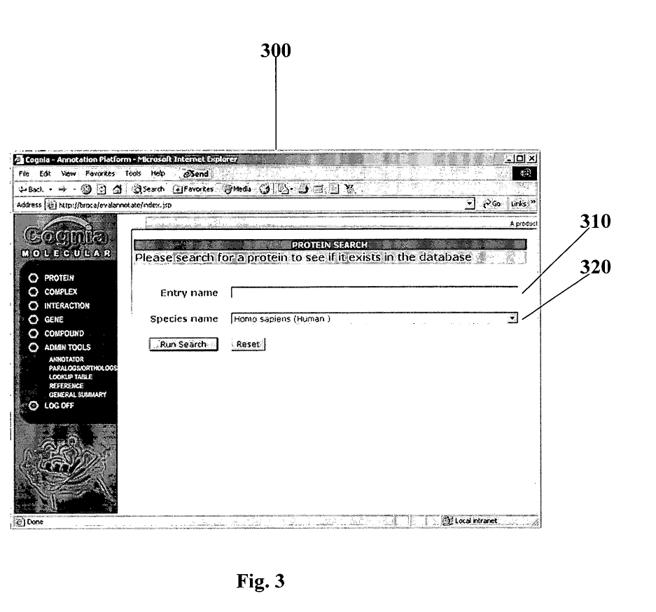

FIG. 3 illustrates an exemplary screen shot of an embodiment of an annotation module and a preliminary search page 300. A user may enter an entry name (selected entry) 310 that is the focus of an annotation. An initial search is conducted to verify that the selected entry 310 is not already part of the database. In order to further refine the initial search, a user may select a particular species containing the molecule in pull-down menu 320.

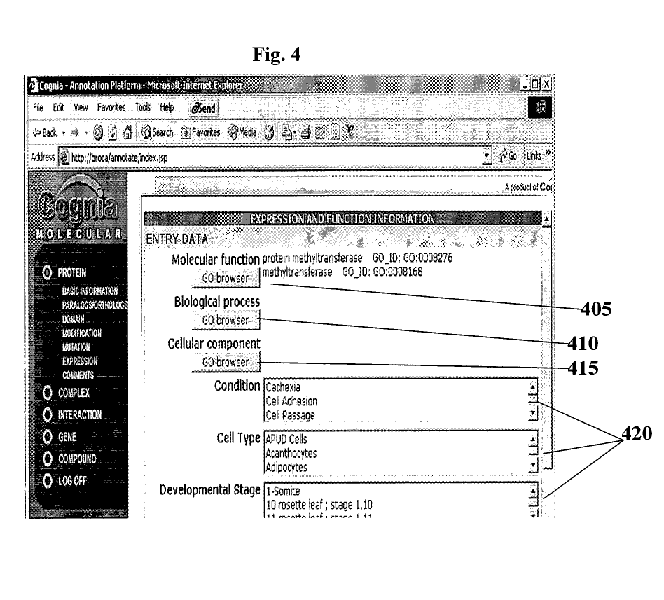

The Cognia Molecular™ annotation module allows for the entry of biological and chemical data, as well as the association of the entered data with a database entry. As illustrated in a universal hierarchy browser in FIG. 4, a molecular function term 405 has been annotated. Alternately, a user could annotate aspects of a data entry's biological process 410, and/or cellular component 415. In this embodiment, the universal browser allows a user to associate at least three attributes 420 with a selected data entry. In the illustrated embodiment, the annotation module is used with the GeneOntology (“GO”) consortium codebase of terms. In one embodiment of the invention, the database system can be used to annotate over 100 different molecular attributes, from proprietary and open-source origins.

Another component module of the Cognia Molecular™ system involves an ontology browser for finding, selecting, and adding terms from inside an ontological system. These terms, in turn, are then used as entry attributes, or flags associated with data entries inside the system and its content.

Ontology Browser and Annotation Utility

Ontologies are standardized vernacular relationships, organized in a rule-based, hierarchical way, whereby terms are defined by a scientific community as a group of relevant semantic descriptors. Without an accepted ontological system, databases can be made. However, they are extremely difficult to integrate with other information systems or data from other sources without some type of standardization. Cognia Molecular™ implements ontologies created from references such as, the Gene Ontology (GO) Consortium and the National Library of Medicine's Medical Subject Headings (MeSH) list. These world standards are the result of consortia of scientists deciding on precise definitions of terms for descriptive cellular, molecular, and physiological biology.

The ontology browser exists as a utility inside the data capture system and is used by a human annotator to add detail to a data entry. Ontologies described herein provide standardized descriptive terms related to aspects of chemical, biological, molecular research such as, but not limited to: protein function, cellular process, sub-cellular location, phylogenetic taxonomy, and disease association. The ontology browser allows the controlled incorporation and association of one or more terms with a database entry. These terms allow the tagging of information and thus, allow the use of searches to retrieve that information through user-defined searches. This invention facilitates the attachment of standard terms to data being entered into a relational database for data entries corresponding to proteins, genes, compounds, complexes and interactions between data entries.

The ontology browser may be used to append reference citations to facts or alternately assertions annotated by human curators to be associated with data entries in the database. Without such references, the database utility and veracity is severely impaired, because researchers require data to be supplied by a credible reference before it can be relied on. The browser polls websites, archives and other scientific resources containing reference material presented in a standard format. To enter data into the database so that other data systems may use the data, the system implements controlled standardized vocabularies from such entities as the GeneOntology consortium, or the Medical Subject Headings list, or other standardized vocabularies. These terms allow the vernacular of biology to be related and controlled in ways that other databases are able to process. Other ontology-related library systems used by Cognia Molecular™ include, but are not limited to, pFAM (protein family) terms, cell type lists from the Cell Line Database and American Type Culture Collection, CDDB, UMLS, SMIRK, SMILES, CD-SMART, SBML, CellML, and open source compound nomenclature from the National Cancer Institute and Daylight technologies. In certain instances, the system implements customized terms, for example, to describe names of research experiments used to demonstrate an empirical fact to be annotated within the system. Depending on the actual embodiment, these term libraries and ontological systems may be configured and accessed by a user in pull down menus (Java based combo boxes or scroll boxes). Alternately, such terms may be embedded in navigation browsers.

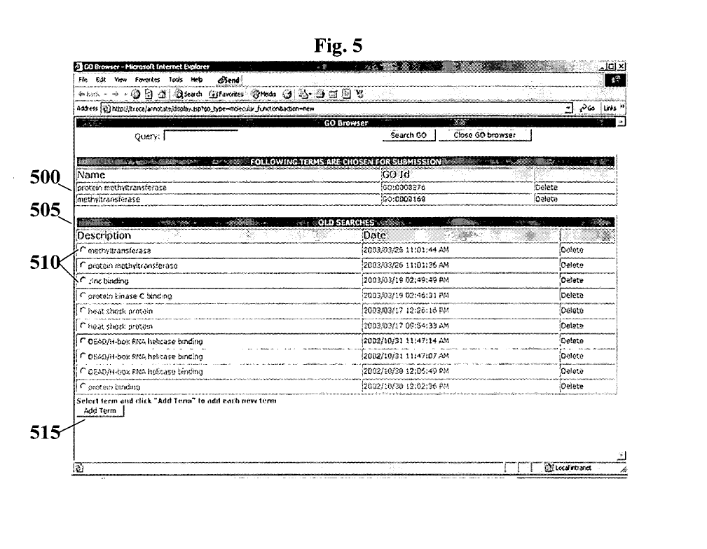

FIG. 5 illustrates an exemplary screen shot of search results associated with an ontology browser's search engine for an embodiment of the present invention. The figure illustrates the current search term 500, as well as search results from previous searches 505 in tabular format. Potential annotation terms are selected by the use of radio buttons 510 embedded in the ontological search browser for entering biological terms into a database. An indefinite number of terms may be appended to a selected database entry. Browser button 515 allows a user to save a term by “adding” it to the list.

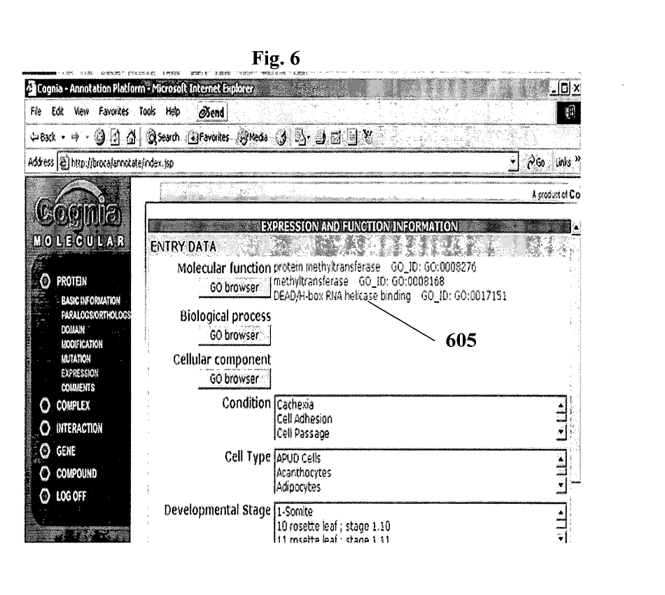

FIG. 6 is an exemplary screen shot associated with the process of adding attribute terms to the annotation through the ontology browser. The figure illustrates the process of “adding” a term. Specifically, as illustrated “DEAD/H-box RNA helicase” binding term 605 has been added to the annotation shown in FIG. 4 and saved in the database.

Terms in the ontologies may be selected from either public or private sources. Such terms are used in a data annotation system that can also be used to create biological databases. The browsers are embedded into a web application and are launched when the user imports data related to a biological process into the database. The ontological component of a relational databasing system is necessary to facilitate viable research due to the complexity associated with the chemical, biological, and molecular elements involved, and the need for standard ontologies that describe similar aspects of cellular and organismal processes. No other data capture system for biological databases implements embedded browsers to house links to readily available scientific resources on the internet and inside the firewall of a user's system, as in the Cognia Molecular™ system.

The ontology browser is a module-based utility for streamlining the capture of biological information as defined by ontological systems. Such systems are necessary for the controlled development of biological databases and provide a standard vernacular whereby such a database may become viable for scientific research resources. Ontologies are not for organizing human thinking and natural intelligence (NI) per se, but rather are highly complex, hierarchical, binary parent-child systems of treed relationships whereby detailed processes, such as cell biology, can be finely subdivided into component logic for automated processing and analysis by artificial intelligence (Al) systems.

The ontology browser is used as part of the Cognia Molecular™ system's user interface when a new ontological term is to be annotated for a database entry. The browser opens, allows a search of the ontologies offline or online and encourages a selection of the ontological term to be appended to the data entry. In an embodiment, the user is presented with the option to save the data. Closing the browser ends the ontological annotation session and the system may revert back to the data entry system. The on-demand browser-in-browser system of CM™ is web enabled, platform agnostic, and universal for all descriptive hierarchical ontologies.

One advantage of the ontological browser system with an annotation module, as opposed to a pull down (“flat”) menu system, is that the ontology browser may be implemented with dynamic upkeep of the ontology by an external agency. This enables the development of the ontological system consistent with the content of the database implementing a universal browser. Accordingly, the invention achieves a dynamic system. Moreover, it guarantees that the user-annotated terms are placed in the correct relational database structure to facilitate advanced Boolean searching capabilities within the system.

The ontology browser enables the use of any ontological system with a hierarchical system of organization. By finely atomizing the biology parameters needed to describe, discuss and research the cell biology and pharmacology of these systems, greater efficiency can be achieved with regard to research information management. An embodiment of the present invention relies on a relational database, platform-general software code proprietary novel semantic lexicons, and universal ontologies.

Reference Manager Annotation Utility

Many types of data processed in the Cognia Molecular™ system are associated with reference tags to indicate the resources that provide the information saved by the annotation pages. This ensures that the data is accurate and is extracted from a reliable source. Implementing reference tags enables a user to trace data inception. Thus, a reference manager has been implemented to import reference and citation data, such as PubMed information. This invention allows a Cognia Molecular™ user to annotate facts in database entries and to append source references to those annotations. Utilizing software scripts, the browser described can automatically extract critical details about those references. This reference manager can be used on any web accessible or intranet data source that is structured according to a standard protocol. The referencing system is web-enabled, platform agnostic, universal for all references and is implemented within the Cognia Molecular™ system.

Disclosed herein is one embodiment of such reference manager, implemented as a medical reference management system. An annotated database resource should reference facts it has aggregated into its corpus. As related to this embodiment, a web-based system is described enabling a method for extracting reference matter from public and proprietary resources. The system is capable of extracting data from regularly structured files from the internet or a local network, as well as enabling a method to enter custom data from unstructured sources, such as anecdotes, seminars, public speeches, journal abstracts, and personal communications. A significant use of the reference parsers described herein involve the use of the medical database “PubMed” and its vast array of articles describing biological and chemical research related to cellular and molecular biology. This utility requires the incorporation of the power of computing and software scripts in aiding the speed and accuracy of data entry in large databases, where a human annotator would have too much information to manually parse individual entries.

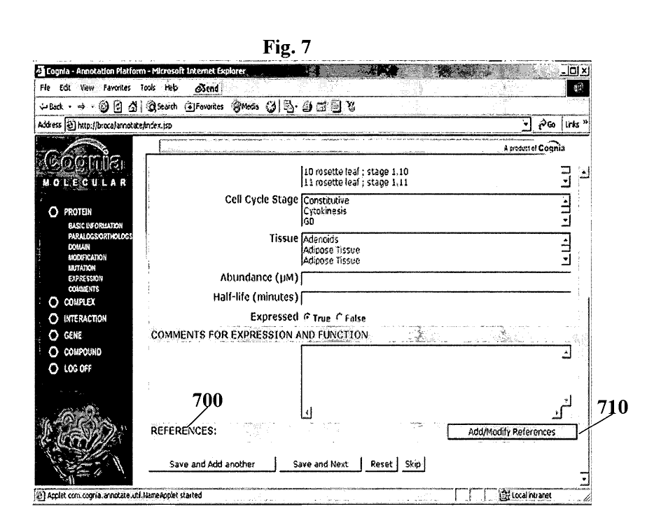

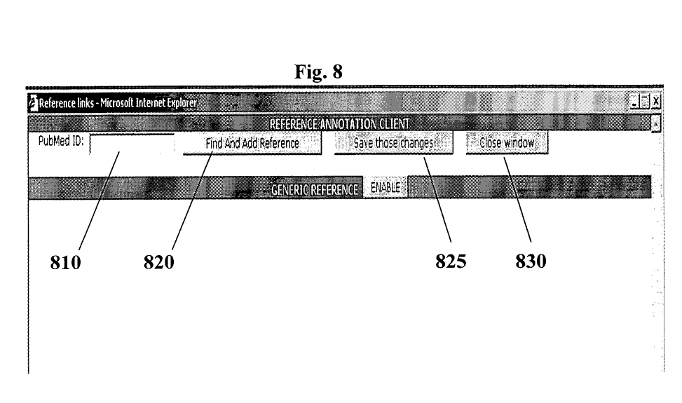

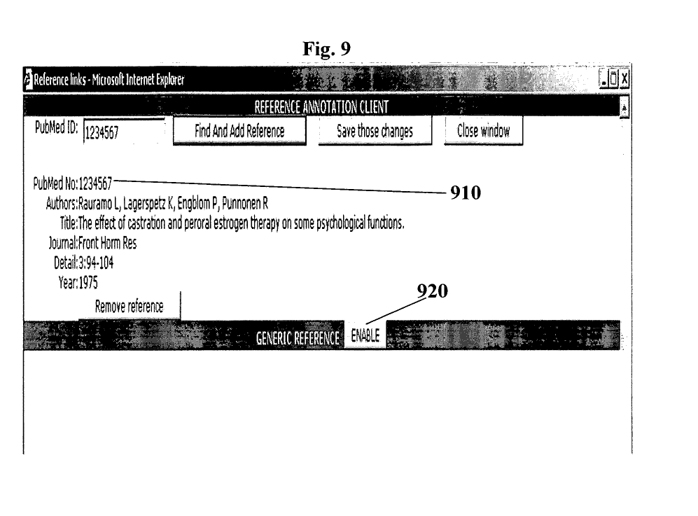

FIG. 7 illustrates the reference manager 700 utility. The embodiment illustrated is an example capable of interacting with the National Library of Medicine's PubMed database. When entering information about a molecule into the relational database, the user:
1) launches a new browser window with a button termed “Add/Modify References 710.”
2) This in turn opens a window shown in FIG. 8, where a user adds a PubMed identification number to the box termed PubMed ID 810; and
3) searches for the reference details using a “Find and Add reference” button 820.
4) The reference manager responds to the user request by searching the National Library of Medicine databases and acquires specific bibliographic information.
5) When the corresponding data has been retrieved, the user may save those changes to the entry by selecting the button, “save those changes” 825.
6) When the user is done adding references, the user may press the “close window” 830 button and proceed to step 7.
7) The user is returned to the original screen wherein the new reference is seen and can be verified 910, shown as in FIG. 9. It is to be understood that the verification 910 is not limited to the illustrated step. The system may verify the spelling of attributes within Cognia Molecular™ database, stored libraries, checking external database identification numbers against database tables with specific formats of identifiers. In the network builder module verification may include calculating molecular weights by summarily adding the grams per mole of the totality of amino acids and generating such a molecule specified by its attributes; calculating protein and gene sequences by summing the number of symbols in their length as defined by standard biopolymer nomenclature.

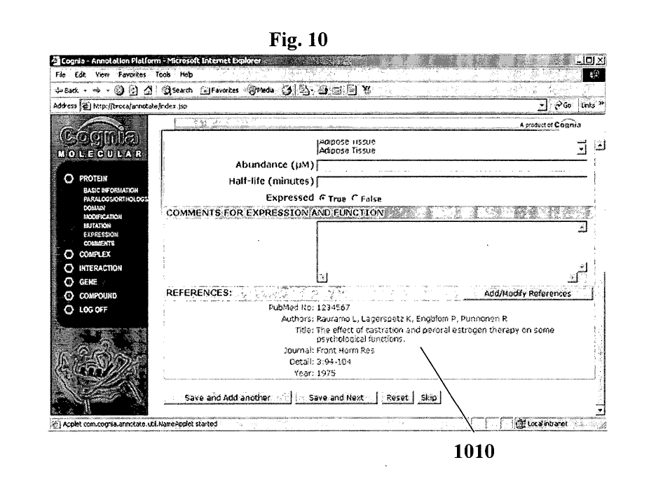

As illustrated in FIG. 10, the saved reference information 1010, provides credibility to the data saved by the annotation module for the particular protein's annotation. It is to be understood that the Cognia Molecular™ system may interact with any other data source comprising structured or unstructured data.

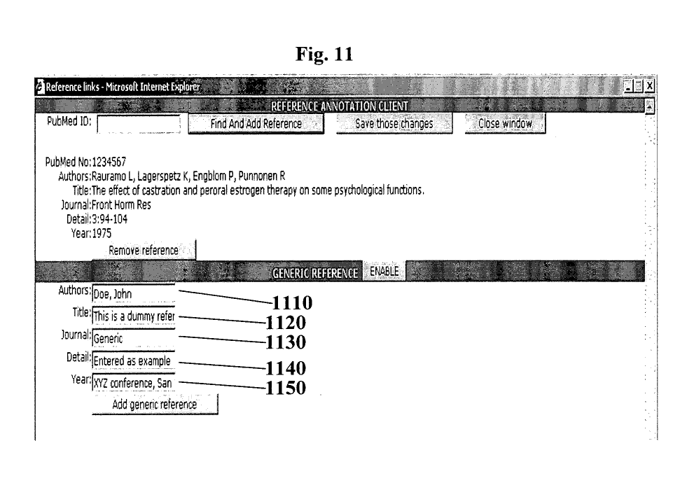

Alternately, FIG. 9 illustrates a “Generic Reference” button 920. This aspect of the present invention allows a user to manually input reference information. Exemplary general reference data is shown in FIG. 11. A user may enter data including, but not limited to the author of a reference 1110, title of a reference 1120, the journal the reference was published in 1130, miscellaneous details 1140, and/or the date of the reference 1150.

Network Builders

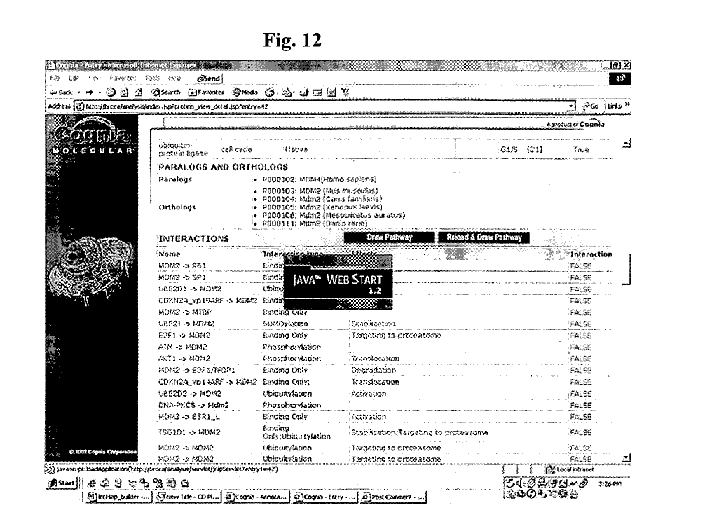

FIGS. 12-15 illustrate one component module of the Cognia Molecular™ system enabling a user to create graphical representations of the cell biology interactions in a network builder module from data stored within the database. To this end, the network builder module is an interaction-building Java application, which creates a molecular interaction diagram (a map), comprising symbols that correspond to a selected data entry's attributes. A network builder provides an easy-to-read graphical representation of interactions between data entries and their corresponding characteristics. Generally, the cellular and molecular interaction data and characteristics are too complex to be addressed by tables, since such interactions often have many features and attributes. The CM™ network builder is integrated with the database schema and in one embodiment of the invention refers to data from at least 140 specific data tables defining various attributes of the data entries.

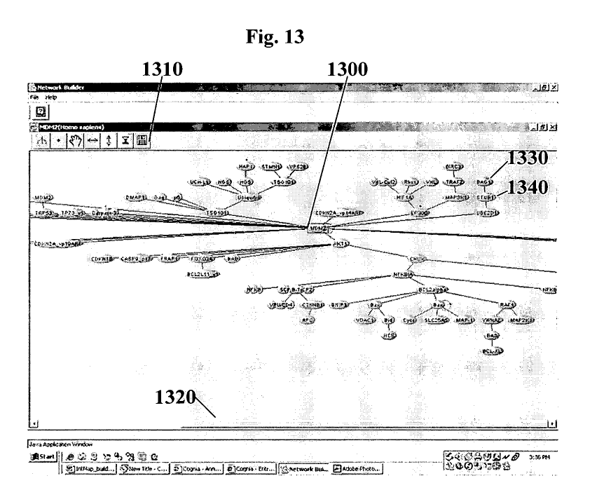

The network builder module is a software utility in CM™ that can graphically map out molecular interactions of a cell biological or a chemical network as shown in FIG. 13. Exemplary graphical representations may include, but are not limited to views of protein interaction networks, cellular positions of potential and existing drug intervention, the display of potential protein drug targets, the regulation of these networks by the members involved.

FIGS. 12-15 also illustrate an example based on data entered into the CM™ system using the annotation module. It is to be understood that the network builder can interact with other relational databases with data stored in normalized or regularized structures, through minor changes to its software code. Also, it is to be understood that although, the illustrated example only shows proteins, the network builder can work with any type of entry in the CM™ system, including protein, gene, complex or compound entries.

The network builder provides a user-friendly interface for a user to interact with the graphical representations illustrated as symbols 1300 in FIG. 13 corresponding to data entries. The graphical representation may be modified by a user to focus on specific interactions through the use of control panel buttons 1310 displayed along the top of the graphical representation. A user may resize the graphical representation to focus on the symbols representing data entries of interest with bar 1320. Alternately, a user may delete symbols associated with non-relevant data entries to focus upon specific data entries. Entries are depicted as nodes 1330 and interactions as lines 1340.

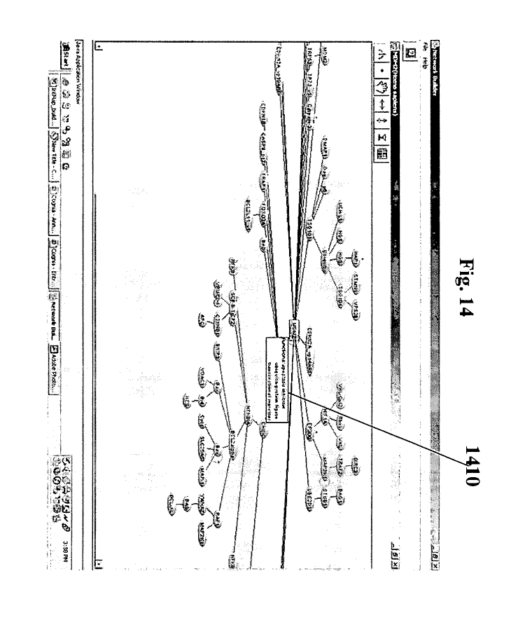

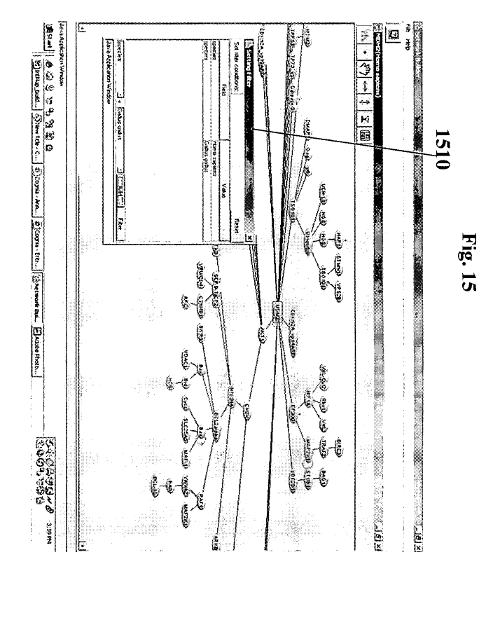

In an embodiment illustrated in FIG. 14, certain characteristics associated with a given symbol may be displayed in another display window 1410 or 1510 in FIGS. 14 and 15, respectively, when a user selects the corresponding symbol. Further, in one embodiment, the user may filter the displayed symbols based on characteristics of the data entries, their interactions, and/or attributes saved by the Cognia Molecular™ system.

Users may filter symbols in the directed graph, using any of the advanced search attributes discussed below. It is to be understood that the invention is not limited to the advanced search attributes described herein. Moreover, in addition to shading, network nodes may be screened, eliminated, expanded or withdrawn.

Advanced Search Algorithms

Another aspect of the Cognia Molecular™ system involves advanced software that searches the chemical, biological, and/or molecular information created using the functionalities described above. The advanced search module uses Boolean search operators and allows recombination of previously executed advanced searches. The module may be configured to store information based on the user identity. Search results may be combined, stored, deleted, reassessed and used in other searches. In one embodiment of the present invention, users of the search facility can access any of 2000 descriptive tags that describe information about the database “entries” by using a series of entry forms and standardized ontological menus. It is to be understood that database entries may include, but are not limited to proteins, genes, bioactive compounds, complexes of these molecules and the interactions between them. The module enables a user to conduct an extremely detailed search of molecules based on their individual properties and attributes. The advanced search algorithm is an extension of the use of ontologies, and implements an exhaustive list of descriptive terms. In an embodiment of the present invention, the advanced searching module implements a database schema that uses tables and relational structures, wherein inherent relationships and interactions between data entries are provided by the nature of descriptive biology.

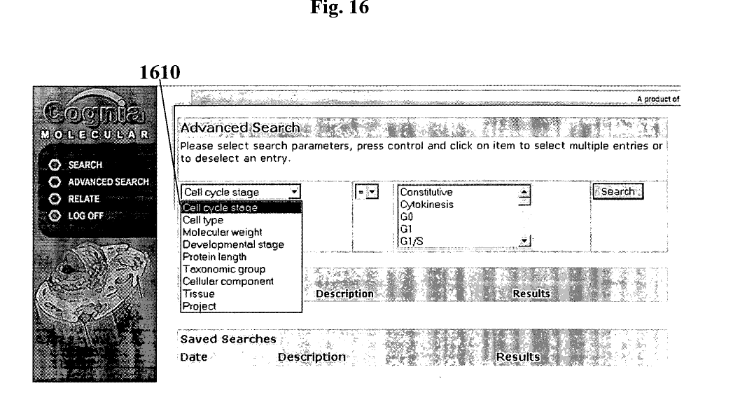

When a user of said computer system wishes to retrieve a database entry using the advanced search functionality based on its attributes, the user:
1) opens Cognia Molecular™;
2) selects the search term from the first menu;
3) views a new set of terms which define the scope of only those search parameters as shown in FIG. 16;
4) selects the search term and executes the query.

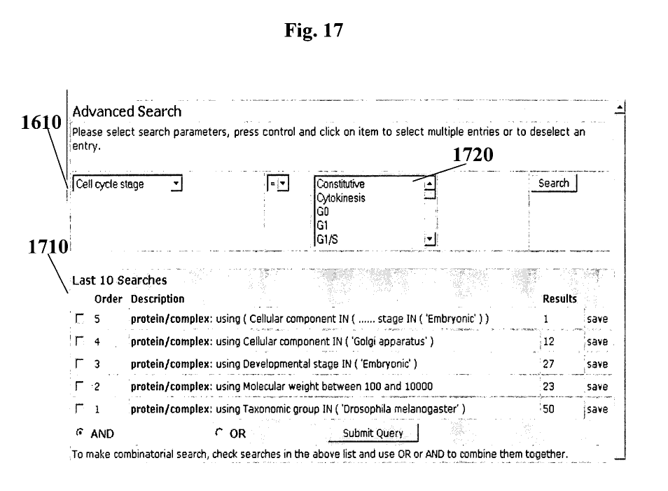

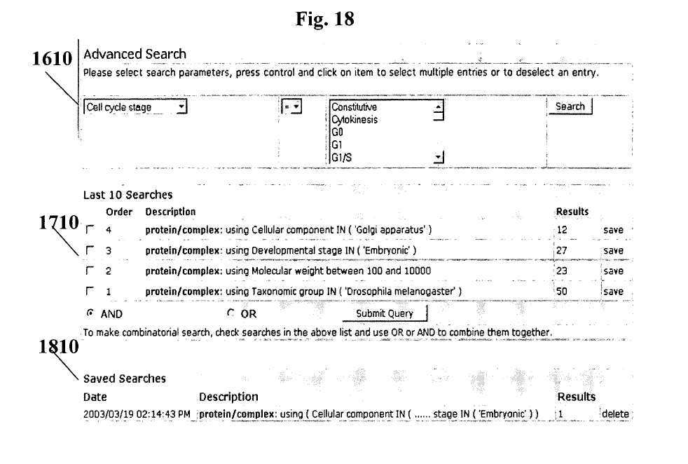

The “fields” or characteristics associated with a data entry that are searched are critical to value and efficacy of such a research tool. Generally, the search fields are based on attributes of the molecule entries present in Cognia Molecular™. It is to be understood that examples of search field parameters may be defined as, but are not limited to terms associated with an entry's cellular and/or biological characteristics. FIGS. 16-18 illustrate various aspects of the advanced search module, including search parameter selection 1610 and further subterm specification for a search as shown by reference numeral 1720. The system also includes both a listing of the last 10 executed searches 1710, as shown in FIG. 17 and a listing of saved searches 1810 shown in FIG. 18.

In an exemplary listing of search subterms associated with the term “Cell Cycle”, a user may search within such characteristics including, but not limited to such specific Cell Cycle subdivisions 1720 as:
Constitutive, Cytokinesis, G0, G1, G1/S, G2, G2/M, Meiosis I-Cytokinesis I, Meiosis I-Metaphase, Meiosis I-Prometaphase, Meiosis I-Prophase, Meiosis I-Telophase, Meiosis II-Metaphase, Meiosis II-Prometaphase, Meiosis II-, Meiosis II-Telophase, Meiosis-Cytokinesis II, Meiosis-all, Mitosis-Anaphase, Mitosis-, Mitosis-Prophase, Mitosis-Telophase, Mitosis-all, S, Terminal.
   Further exemplary advanced search terms 1610 may include, each with their own subterms:
Cell cycle stage, Cell type, Molecular weight, Developmental stage, Protein length, Taxonomic group, Cellular component, Tissue, Annotation Project, Molecular function, Chemical, Disease, Network, Pathway, pI, pKa, boiling point, melting point, domain, Biological Process.

Alternately, the search may be directed to a scientifically topical project in question such as “catabolism,” “test project,” or “molecular trafficking.” It is to be understood that the example discussed above illustrates simply representative terms and actual search terms may include a vast range of biological, chemical and/or molecular descriptive characteristics. The advanced searching module enables biologists to search based on.biological characteristics of a given cellular system. By finely atomizing the parameters needed to discuss and research the cell biology and pharmacology of these systems, better research information management can be achieved.

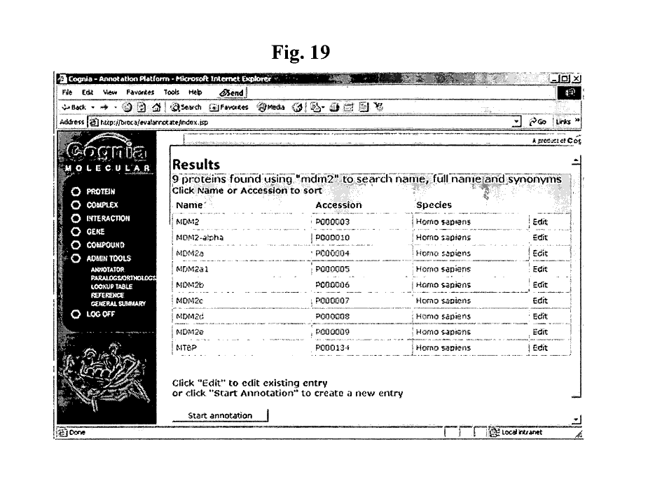

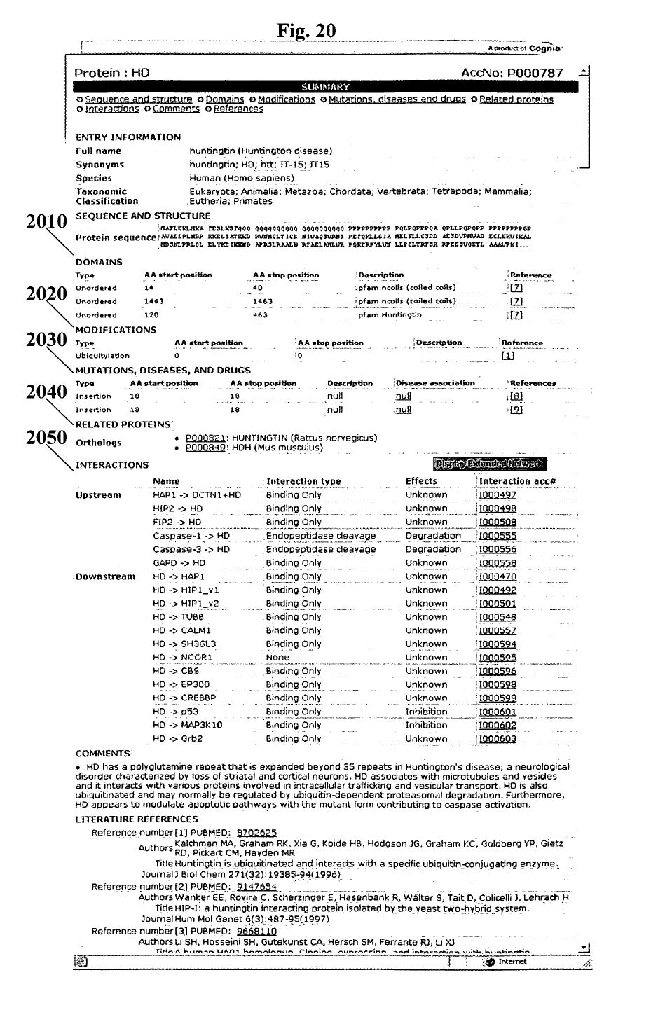

FIG. 19 illustrates an exemplary screen shot displaying results obtained from an advanced search. FIG. 20 illustrates an exemplary output table that corresponds to a data entry created using the components of the Cognia Molecular™ database system described above. More specifically, FIG. 20 illustrates domains of proteins 2010, their post-translational modifications 2020, their mutations 2030, proteins similar in sequence 2040 and the interactions of the molecule 2050.

Structural Search Tools

The advanced searching module described above provides a broader search capability by incorporating other types of search algorithms. This results in a more robust search and retrieval of protein, nucleic acid and chemical structures. Through archiving based on features of chemicals, such as, but not limited to, functional groups, chemical moieties, and/or synthetic pathways, the Cognia Molecular™ system is able to retrieve entries including ketones, aldehydes, sulfydryls, alcohols, pyrolles, and other types of chemical groups or species.

Portions of chemical compounds may be searched by using text-based or SMILES-based searches for substructural components. Each element defined above may be specified in such a manner.

Batch Data Importers

The utility of Cognia Molecular™ is further enhanced by the ability of a user to incorporate a vast range of a user's existing in-house data into the system. The CM™ system is configurable to process and incorporate such pre-existing research data with data processing algorithms and become integrated with other data entries included in the Cognia Molecular™ system database.

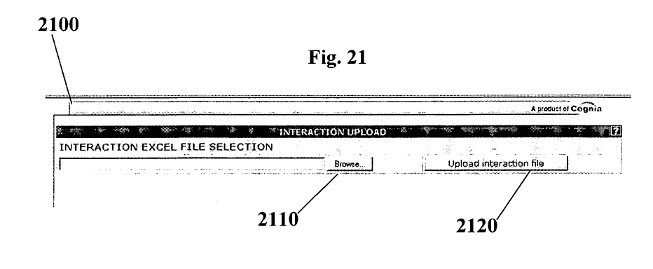

Accordingly, a batch data importer module converts such batch data via high throughput methods for incorporation into the Cognia Molecular™ system. The batch data importer module may parse and incorporate user selected tables of information, wherein a user may specify table attributes for import and conversion. By way of example only, the batch data importer may incorporate a pre-existing user-developed list of proteins that interact with each other into a Cognia Molecular™ database, by specifying the translation protocol from the current data format to a Cognia Molecular™ system format. Specifically, in an embodiment of the invention, protein A becomes “component A” which “interacts with” protein B, or “component B”. FIG. 21 shows a representative user interface for the interaction loader 2100. Users may search their local node for a tabular file 2110 and load it into the system 2120.

Cognia Molecular™ can incorporate data formats including, but not limited to excel-type spreadsheets, delimited text files, comma separated files, and other standardized data formats such as marked up text (XML, HTML, dHTML, SBML, CellML) and other relational database sources. Such data is imported into the relational structure of the database automatically, using a simple loader interface 2100 as shown in FIG. 21. The interface includes a browser 2110 for the user generated files and an “upload” button 2120. The batch loader relies only on a regular structure of the data to be incorporated. Through use of the batch loader, the user can specify the target and destination database tables and import the batch data directly into the relational database tables of the invention. The import module thus removes human error and facilitates a more efficient data import. Specific batch loaders may also include those used expressly for the import of dichotomous/binary interaction data. Such data is simpler and does not necessarily need to be elaborated upon.

Administrator Control Applications.

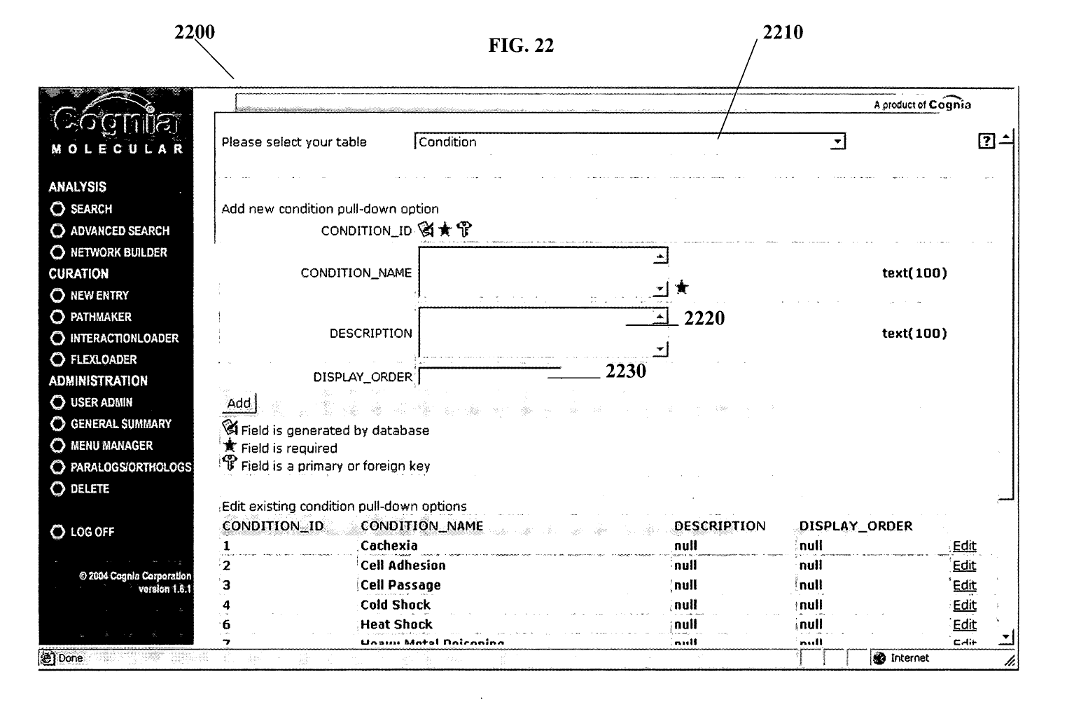

A further aspect of the present invention involves administrator control (Admin functions) for the implementing the annotation system. Using the Administration functions present in a navigation bar, it is possible for a system administrator, “superuser,” to define accessibility and content features associated with the system. It is to be understood that such features may include, but are not limited to limited functionality privileges, user start and stop dates, and search menu content. Additionally, the admin panel allows the superuser to define paralogs, orthologs, access privileges, passwords, usemames, restricted access based on IP address and other system access parameters, without necessarily having to access the relational tables using a browser or schema manager. One exemplary manager for curation menus is shown in FIG. 22. Users may select the database table 2210, add descriptions 2220, and control the appearance in the parent table 2230.

The many features and advantages of the present invention are apparent from the detailed specification, and thus, it is intended by the appended claims to cover all such features and advantages of the invention which fall within the true spirit and scope of the invention. Furthermore, since the embodiments described above are exemplary, numerous modifications and variations will readily occur to those skilled in the art, and the invention should not be limited to the exact construction and operation illustrated and described herein.

1. A method for curating, researching and analyzing scientific data and information comprising:
receiving in a central server a search request from curation pages on a user node;
correlating the search request to at least one database of protein, gene, complex, compound, and interaction data entries;
searching the at least one database;
creating a search result listing of published resources from the at least one database;
extracting data from published resources related to the search term;
creating a set of database entry attributes based on extracted data related to the database entry;
associating the set of attributes with the database entry and transmitting the set of attributes to the database for storage;
creating a graphical representation of data associated with the data entry; and
transmitting the graphical representation to the user node for subsequent display to a user.

2. The method in claim 1, further comprising:
implementing a web-based annotation module and analysis module which connect to a remote database server.

3. A method for transmitting search requests to a central server, further comprising:
receiving user data in a user node associated with user a logon,
transmitting said logon data to the central server, wherein said central server authenticates the user data;
receiving authorization from the central server allowing the user node to provide the user with access to the central server;
requesting a list of search terms related to at least one of biological, chemical and molecular compounds from the central server;
selecting a search term, wherein said search term forms the basis for complex searching of a database that stores data entries from at least one of chemical, biological, or molecular research fields;
creating a complex search request identifying the selected search term on the database for performing the complex search;
transmitting said complex search request to the central server;
receiving a complex search response from the central server, wherein said user node displays results produced by complex searching of the database, in accordance with the complex search response.

4. A method for querying a database of data entries from chemical, biological, or molecular research fields comprising:
receiving a complex search request from a central server, wherein said complex search request includes data identifying a search term selected from at least one of chemical biological, or molecular research fields;
searching the database for the search term specified in the complex search request;
creating a search response based on results of the database search, wherein said search response includes search term status in the database at the time of the database search and any associated data entry attributes;
transmitting said search response to the central server.

5. A method of implementing server side functionality, wherein said server side functionality comprises:
receiving a search request from a user node;
transmitting data between a server and a chemical, biological, and molecular compound database; and
creating a data message describing the interactions of molecules through the use of database tables;
transmitting the data message to a user node for subsequent display to a user.

6. The method described in claim 5, wherein the interactions of molecules corresponds to the interactions of proteins.

7. The method described in claim 5, wherein the interactions of molecules corresponds to the interactions of genes.

8. The method described in claim 5, wherein the interactions of molecules corresponds to the interactions of compounds.

9. The method described in claim 5, wherein the interactions of molecules corresponds to the interactions of complexes of molecules.

10. The method described in claim 5, wherein the interactions of molecules corresponds to the interactions of molecular moieties.

11. The method of claim 5 further comprising:
basing the search request on attribute terms from at least one of an ontology or semantic lexical library selected from a plurality of sources;
wherein database table terms describe biological, chemical, or molecular attributes of the entries; and
conducting a search of ontological data in real time.

12. The method of claim 11 further comprising:
searching terms from a static or dynamic list.

13. The method of claim 11, wherein the attributes are related to entry species, developmental stage, cell cycle stage, or a project in question.

14. The method of claim 11, further comprising:
coordinating orthology and paralogy groupings by a software script.

15. A method of maintaining data entry attribute integrity comprising:
entering biological, chemical, or molecular characteristics corresponding to an entry as at least one attribute;
verifying the spelling of said attribute, external database identification numbers, molecular weights, protein and gene sequences, molecular lengths as the at least one attribute is entered;
defining a variable corresponding to whether reciprocal data associated with an entry is commutative;
determining whether a search request has been previously entered; and
parsing public databases for protein and genetic information.

16. The method claim of 15, wherein:
the protein and genetic information includes a name corresponding to a data entry.

17. The method claim of 15, wherein:
the protein and genetic information includes a synonym.

18. The method claim of 15, wherein:
the protein and genetic information includes a sequence.

19. The method claim of 15, wherein:
the protein and genetic information includes a weight.

20. The method of claim 15, wherein:
the protein and genetic information includes a molecular mass.

21. The method of claim 15, wherein:
the protein and genetic information includes an isoelectric point.

22. The method of claim 15, wherein:
the protein and genetic information includes a length.

23. The method of claim 15, wherein:
the protein and genetic information includes a percent AT content.

24. The method of claim 15, wherein:
the protein includes a percent amino acid analysis.

25. The method of claim 15, wherein:
the protein and genetic information includes any database reference ID.

26. The method of claim 15, further comprising:
defining functionally or genetically homologous relationships of genes and gene products by creating automated orthology groupings.

27. The method of claim 15, further comprising:
searching data entries present in the database using an advanced search utility;
navigating external databases which contain related data;
entering data present in external databases as hierarchies; and
entering genetic and protein data into a bioinformatic database obtained from searching said databases with an advanced search utility.

28. The method of claim 27, further comprising:
implementing a graphical user interface allowing a user to define hypothetical relationships between entries in the database.

29. A method for searching ontological data comprising:
transmitting data requests from a local node to external ontological databases;
conducting a search of external ontological databases from said local node based on data requests, wherein the data request uses search terms related to biological, chemical, or molecular fields;
saving the search terms on the local node; and
displaying results of the search on a local node.

30. The method of claim 29 wherein:
the external ontological databases are warehoused public ontological systems.

31. The method of claim 29 wherein:
the external ontological databases are private hierarchical ontological systems.

32. The method of claim 29, wherein the search terms include disease associations, OMIM (Online Mendelian Inheritance) terms, Medical Subject Headings (MeSH) terms, and the GO terms: Molecular Function, Cellular Process, and Subcellular component.

33. The method of claim 29, further comprising:
screening search terms available from ontological hierarchies;
including previously saved search terms in an immediately viewable menu;
selecting from a listing of search results;
appending ontological terms to a specific entry; and
saving appended ontological relationships into the specific entry.

34. A method for providing references to database entries comprising:
extracting information from chemical, biological, or molecular resources, for a molecular attribute or a specific database entry selected by a curator, wherein said curator associates the extracted information with the molecular attribute or database entry; and
creating an annotation between the extracted information with the corresponding resource associated with the extracting information.

35. The method of claim 34, wherein:
the extracted information is at least one of facts, assertions, hypotheses related to the molecular attribute or specific database entry.

36. The method of claim 35, wherein:
the chemical, biological, or molecular resources include public archives, private electronic sources, electronic conference abstracts, and electronic marketing disseminations.

37. The method of claim 34, wherein:
the extracted information includes sources of sequence data regarding proteins, genes, and gene loci.

38. The method of claim 34, wherein:
the annotation is a hyperlink correlating the extracted information associated with the molecular attribute or database entry and the corresponding resource.

39. The method of claim 34, further comprising:
initiating the extracting process based on a user selection.

40. The method of claim 39, further comprising:
searching National Library of Medicine for journal article, book, or abstract PubMed identification numbers.

41. The method of claim 40, further comprising:
automatically importing to a user node all reference information collected from the National Library of Medicine with corresponding PubMed identification numbers.

42. The method of claim 34, further comprising:
creating a specific annotation for resources that are not structured in accordance with a predetermined resource.

43. The method of claim 34, wherein:
the chemical, biological, or molecular resources include electronic copies of personal communiqué, abstracts, newspapers, flyers, microfilms, microfiche or websites.

44. A method for analyzing database entries comprising:
saving interaction characteristics associated with chemical, biological, or molecular entries within a relational database, in said entries; and
creating a graphical representation illustrating said interaction characteristics for at least one selected entry, wherein the graphical representation includes symbols representing molecules and lines between database entries indicating molecular interaction.

45. The method of claim 44, further comprising:
recreating the graphical representation to reflect modifications to the interaction characteristics, wherein said recreation is initiated by a user selecting a refresh option that loads additional data to the utility.

46. The method of claim 44, wherein:
the symbols represent molecules including proteins, complexes, drug compounds, or genes.

47. The method of claim 44, wherein:
the visual representation includes interaction characteristics extracted from at least one chemical, biological, or molecular resource.

48. The method of claim 47, wherein:
at least one chemical, biological, or molecular resource includes molecular purifications, gel filtration data, two hybrid experiments, surface plasmon resonance assays, measurements using electromagnetic radiation, indirect kinetic experimentation data, and many other types of molecular biological or chemical data.

49. The method of claim 44, further comprising:
manipulating a control menu associated with the graphical representation enabling a user to customize the graphical representation appearance and control what is displayed.

50. The method of claim 49, wherein:
the graphical representation displays all interactions connected with a single central molecule.

51. The method of claim 49, wherein:
the graphical representation limits the interaction display to the interactions of the central molecule and symbols representing directly connected data entries.

52. The method of claim 44, wherein:
a user manipulates a virtual hand to navigate the graphical representation.

53. The method of claim 44, further comprising:
resizing the graphical representation along vertical or horizontal axes.

54. The method of claim 44, further comprising:
saving the graphical representation as a file.

55. The method of claim 44, further comprising:
printing the graphical representation.

56. The method of claim 44, further comprising:
superimposing a new graphical representation displaying additional attributes or information associated with the database entry in response to selecting a system option associated with a specific data entry.

57. The method of claim 44, further comprising:
correlating line thickness between symbols on the graphical representation with the experimental reliability of the annotated data entries.

58. The method of claim 44, further comprising:
hiding or deleting symbols on the graphical representation.

59. The method of claim 44, further comprising:
rotating the graphical representation.

60. The method of claim 44, further comprising:
reproportioning the graphical representation in accordance with a user-specified reproportioning ratio.

61. The method of claim 44, further comprising:
reproportioning the graphical representation in accordance with a predetermined reproportioning ratio.

62. The method of claim 44, further comprising:
searching the graphical representation for node symbols corresponding to database entries, using attributes associated with said database entry.

63. The method of claim 44, further comprising:
splitting the graphical representation into sub-graphical representations, wherein said sub-graphical representations correspond to specific database entries.

64. The method of claim 63, wherein the specific database entries are different species.

65. The method of claim 44, further comprising:
summarizing attributes associated with database entries corresponding to symbols displayed on the graphical representation; and
displaying a summary graphical representation to the user.

66. The method of claim 44, further comprising:
transitioning from the graphical representation to a view of detailed attributes associated with a database entry in response to a user initiated command.

67. The method of claim 44, further comprising:
modifying the graphical representation to display symbols representing database entries with specific attributes in accordance with a user determined set of display-filter attributes.

68. The method of claim 67, wherein the display-filter attributes filter symbols based on database entry attributes comprising:
species, molecular mass, cell cycle expression, developmental stage, taxonomic group, tissue specificity, subcellular location, cell type, experimental condition, molecular function of the database entry, cellular process which the entry is a part, source of the molecule from which it was purified, effect of the interaction on at least one interacting symbol, type of interaction.

69. The method of claim 68, wherein the type of interaction comprises:
database entries binding, inhibiting, or activating interaction.

70. A method for conducting complex searching of database entries, comprising:
compiling a set of predetermined search terms, wherein said search terms correspond to database entry attributes;
receiving a user-created query in accordance with Boolean logical formulation;
saving the user-created query in response to a user-initiated save search command;
searching the database entries in accordance with a complex searching protocol, wherein the predetermined search terms are related to the fields of cellular, biological or physiological scientific research.

71. The method of claim 70, further comprising:
modifying the saved search wherein, the user combines the saved search with previously saved search.

72. The method of claim 70, further comprising:
modifying the saved search wherein, the user deletes a search term from the saved search.

73. The method of claim 70, further comprising:
modifying the saved search wherein, the user adds a search term from the saved search.

74. The method of claim 70, wherein:
the database entry corresponds to relational database schema describing entries in the system, each of which has attributes that are searchable.

75. The method of claim 74, further comprising:
searching data attributes based on the relational database schema.

76. The method of claim 70, further comprising:
creating pull down menus, wherein each menu defines attributes of the database entries corresponding to proteins, genes, compounds, complexes of molecules, or interactions of molecular moieties.

77. The method of claim 76, wherein said pull down menus are configured to be searchable for standardized ontologies.

78. The method of claim 77, further comprising:
removing extraneous material from said pull down menus in accordance with predetermined protocols related to a search parameter.

79. The method of claim 70, further comprising:
associating the saved search with a specific user;
presenting a list of previous saved searches associated with said specific user in response to a user-issued command.

80. The method of claim 70, further comprising:
reordering of said search results according to user manipulation of column header organization buttons.

81. The method of claim 1, further comprising:
importing regularly structured table data automatically, using an interface on a user node.

82. The method of claim 70, further comprising:
importing regularly structured table data automatically, using an interface on a user node.

83. The method of claim 81, further comprising a search facility configured to search the table data on the user node.

84. The method of claim 81, further comprising a tool to import and automatically upload the table data.

85. The method of claim 81, wherein a user can import new database tables altering a system's schema.

86. The method of claim 85, wherein tables from the system are selectable.

87. The method of claim 85, wherein description of the new table field is added.

88. The method of claim 85, wherein the display order of the new data is specified for user output analysis pages.

89. A system for curating, researching and analyzing scientific data and information comprising:
a memory having program code stored therein;
a processor operatively connected to said memory for carrying out instructions in accordance with said stored program code, wherein said program code, when executed by said processor causes said processor to:
receive in a central server a search request from curation pages on a user node;
correlate the search request to at least one database of protein, gene, complex, compound, and interaction data entries;
search the at least one database;
create a search result listing of published resources from the at least one database;
extract data from published resources related to the search term;
create a set of database entry attributes based on extracted data related to the database entry;
associate the set of attributes with the database entry and transmitting the set of attributes to the database for storage;
create a graphical representation of data associated with the data entry; and
transmit the graphical representation to the user node for subsequent display to a user.

90. The system in claim 89, wherein said processor is further operative to:
implement a web-based annotation module and analysis module which connect to a remote database server.

91. A system for transmitting search requests to a central server, comprising:
a memory having program code stored therein;
a processor operatively connected to said memory for carrying out instructions in accordance with said stored program code, wherein said program code, when executed by said processor causes said processor to:
receive user data in a user node associated with user a logon,
transmit said logon data to the central server, wherein said central server authenticates the user data;
receive authorization from the central server allowing the user node to provide the user with access to the central server;
request a list of search terms related to at least one of biological, chemical and molecular compounds from the central server;
select a search term, wherein said search term forms the basis for complex searching of a database that stores data entries from at least one of chemical, biological, or molecular research fields;
create a complex search request identifying the selected search term on the database for performing the complex search;
transmit said complex search request to the central server;
receive a complex search response from the central server, wherein said user node displays results produced by complex searching of the database, in accordance with the complex search response.

92. A system for querying a database of data entries from chemical, biological, or molecular research fields comprising:
a memory having program code stored therein;
a processor operatively connected to said memory for carrying out instructions in accordance with said stored program code, wherein said program code, when executed by said processor causes said processor to:
receive a complex search request from a central server, wherein said complex search request includes data identifying a search term selected from at least one of chemical biological, or molecular research fields;
search the database for the search term specified in the complex search request;
create a search response based on results of the database search, wherein said search response includes search term status in the database at the time of the database search and any associated data entry attributes;
transmit said search response to the central server.

93. A system of implementing server side functionality, wherein said system comprises:
a memory having program code stored therein;
a processor operatively connected to said memory for carrying out instructions in accordance with said stored program code, wherein said program code, when executed by said processor causes said processor to:
receive a search request from a user node;
transmit data between a server and a chemical, biological, and molecular compound database; and
create a data message describing the interactions of molecules through the use of database tables;
transmit the data message to a user node for subsequent display to a user.

94. The system described in claim 93, wherein said processor is further operative to correlate the interactions of molecules to the interactions of proteins.

95. The system described in claim 93, wherein said processor is further operative to correlate the interactions of molecules to the interactions of genes.

96. The system described in claim 93, wherein said processor is further operative to correlate the interactions of molecules to the interactions of compounds.

97. The system described in claim 93, wherein said processor is further operative to correlate the interactions of molecules to the interactions of complexes of molecules.

98. The system described in claim 93, wherein said processor is further operative to correlate the interactions of molecules to the interactions of molecular moieties.

99. The system of claim 93, wherein said processor is further operative to:
base the search request on attribute terms from at least one of an ontology or semantic lexical library selected from a plurality of sources;
wherein database table terms describe biological, chemical, or molecular attributes of the entries; and
conduct a search of ontological data in real time.

100. The system of claim 99, wherein said processor is further operative to:
search terms selected from a combo box from a static or dynamic list;
conduct a search of ontological data in real time.

101. The system of claim 99, wherein the attributes are related to entry species, developmental stage, cell cycle stage, or a project in question.

102. The system of claim 99, wherein said processor is further operative to:
coordinate orthology and paralogy groupings by a software script.

103. A system of maintaining data entry attribute integrity comprising:
a memory having program code stored therein;
a processor operatively connected to said memory for carrying out instructions in accordance with said stored program code, wherein said program code, when executed by the said processor causes said processor to:
enter biological, chemical, or molecular characteristics corresponding to an entry as at least one attribute;
verify the spelling of said attribute, external database identification numbers, molecular weights, protein and gene sequences, molecular lengths as the at least one attribute is entered;
define a variable corresponding to whether reciprocal data associated with an entry is commutative;
determine whether a search request has been previously entered; and
parse public databases for protein and genetic information.

104. The system of claim 103, wherein:
the protein and genetic information includes a name corresponding to a data entry.

105. The system of claim 103, wherein:
the protein and genetic information includes a synonym.

106. The system of claim 103, wherein:
the protein and genetic information includes a sequence.

107. The system of claim 103, wherein:
the protein and genetic information includes a weight.

108. The system of claim 103, wherein:
the protein and genetic information includes a molecular mass.

109. The system of claim 103, wherein:
the protein and genetic information includes an isoelectric point.

110. The system of claim 103, wherein:
the protein and genetic information includes a length.

111. The system of claim 103, wherein:
the protein and genetic information includes a percent AT content.

112. The system of claim 103, wherein:
the protein includes a percent amino acid analysis.

113. The system of claim 103, wherein:
the protein and genetic information includes any database reference ID.

114. The system of claim 103, wherein said processor is further operative to:
define functionally or genetically homologous relationships of genes and gene products by creating automated orthology groupings.

115. The system of claim 103, wherein said processor is further operative to:
search data entries present in the database using an advanced search utility;
navigate external databases which contain related data;
entering data present in external databases as hierarchies; and
enter genetic and protein data into a bioinformatic database obtained from searching said databases with an advanced search utility.

116. The system of claim 115, wherein said processor is further operative to:
implement a graphical user interface allowing a user to define hypothetical relationships between entries in the database.

117. A system for searching ontological data comprising:
a memory having program code stored therein;
a processor operatively connected to said memory for carrying out instructions in accordance with said stored program code, wherein said program code, when executed by said processor causes said processor to:
transmit data requests from a local node to external ontological databases;
conduct a search of external ontological databases from said local node based on data requests, wherein the data request uses search terms related to biological, chemical, or molecular fields;
save the search terms on the local node; and
display results of the search on a local node.

118. The system of claim 117, wherein:
the external ontological databases are warehoused public ontological systems.

119. The system of claim 117 wherein:
the external ontological databases are private hierarchical ontological systems.

120. The system of claim 117, wherein the search terms include disease associations, OMIM (Online Mendelian Inheritance) terms, Medical Subject Headings (MeSH) terms, and the GO terms: Molecular Function, Cellular Process, and Subcellular component.

121. The system of claim 117, wherein said processor is further operative to:
screen search terms available from ontological hierarchies;
include previously saved search terms in an immediately viewable menu;
select from a listing of search results;
append ontological terms to a specific entry; and
save appended ontological relationships into the specific entry.

122. A system for providing references to database entries comprising:
a memory having program code stored therein;
a processor operatively connected to said memory for carrying out instructions in accordance with said stored program code, wherein said program code, when executed by said processor causes said processor to:
extract information from chemical, biological, or molecular resources, for a molecular attribute or a specific database entry selected by a curator, wherein said curator associates the extracted information with the molecular attribute or database entry; and
create an annotation between the extracted information with the corresponding resource associated with the extracting information.

123. The system of claim 122, wherein:
the extracted information is at least one of facts, assertions, hypotheses related to the molecular attribute or specific database entry.

124. The system of claim 123, wherein:
the chemical, biological, or molecular resources include public archives, private electronic sources, electronic conference abstracts, and electronic marketing disseminations.

125. The system of claim 122, wherein:
the extracted information includes sources of sequence data regarding proteins, genes, and gene loci.

126. The system of claim 122, wherein:
the annotation is a hyperlink correlating the extracted information associated with the molecular attribute or database entry and the corresponding resource.

127. The system of claim 122, wherein said processor is further operative to:
initiate the extracting process based on a user selection.

128. The system of claim 127, wherein said processor is further operative to:
search National Library of Medicine for journal article, book, or abstract PubMed identification numbers.

129. The system of claim 128, wherein said processor is further operative to:
automatically import to a user node all reference information collected from the National Library of Medicine with corresponding PubMed identification numbers.

130. The system of claim 122, wherein said processor is further operative to:
create a specific annotation for resources that are not structured in accordance with a predetermined resource.

131. The system of claim 122, wherein:
the chemical, biological, or molecular resources include electronic copies of personal communiqué, abstracts, newspapers, flyers, microfilms, microfiche or websites.

132. A system for analyzing database entries comprising:
a memory having program code stored therein;
a processor operatively connected to said memory for carrying out instructions in accordance with said stored program code, wherein said program code, when executed by said processor causes said processor to:
save interaction characteristics associated with chemical, biological, or molecular entries within a relational database, in said entries; and
create a graphical representation illustrating said interaction characteristics for at least one selected entry, wherein the graphical representation includes symbols representing molecules and lines between database entries indicating molecular interaction.

133. The system of claim 132, wherein said processor is further operative to:
recreate the graphical representation to reflect modifications to the interaction characteristics, wherein said recreation is initiated by a user selecting a refresh option that loads additional data to the utility.

134. The system of claim 132, wherein:
the symbols represent molecules including proteins, complexes, drug compounds, or genes.

135. The system of claim 132 wherein:
the visual representation includes interaction characteristics extracted from at least one chemical, biological, or molecular resource.

136. The system of claim 135, wherein:
at least one chemical, biological, or molecular resource includes molecular purifications, gel filtration data, two hybrid experiments, surface plasmon resonance assays, measurements using electromagnetic radiation, indirect kinetic experimentation data, and many other types of molecular biological or chemical data.

137. The system of claim 132, wherein said processor is further operative to:
manipulate a control menu associated with the graphical representation enabling a user to customize the graphical representation appearance and control what is displayed.

138. The system of claim 137, wherein:
the graphical representation displays all interactions connected with a single central molecule.

139. The system of claim 137, wherein:
the graphical representation limits the interaction display to the interactions of the central molecule and symbols representing directly connected data entries.

140. The system of claim 132, wherein:
a user manipulates a virtual hand to navigate the graphical representation.

141. The system of claim 132, wherein said processor is further operative to:
resize the graphical representation along vertical or horizontal axes.

142. The system of claim 132, wherein said processor is further operative to:
save the graphical representation as a file.

143. The system of claim 132, further comprising:
print the graphical representation.

144. The system of claim 132, wherein said processor is further operative to:
superimpose a new graphical representation displaying additional attributes or information associated with the database entry in response to selecting a system option associated with a specific data entry.

145. The system of claim 132, wherein said processor is further operative to:
correlate line thickness between symbols on the graphical representation with the experimental reliability of the annotated data entries.

146. The system of claim 132, wherein said processor is further operative to:
hide or delete symbols on the graphical representation.

147. The system of claim 132, wherein said processor is further operative to:
rotate the graphical representation.

148. The system of claim 132, wherein said processor is further operative to:
reproportion the graphical representation in accordance with a user-specified reproportioning ratio.

149. The system of claim 132, wherein said processor is further operative to:
reproportion the graphical representation in accordance with a predetermined reproportioning ratio.

150. The system of claim 132, wherein said processor is further operative to:
search the graphical representation for node symbols corresponding to database entries, using attributes associated with said database entry.

151. The system of claim 132, wherein said processor is further operative to:
split the graphical representation into sub-graphical representations, wherein said sub-graphical representations correspond to specific database entries.

152. The system of claim 151, wherein the specific database entries are different species.

153. The system of claim 132, wherein said processor is further operative to:
summarize attributes associated with database entries corresponding to symbols displayed on the graphical representation; and
display a summary graphical representation to the user.

154. The system of claim 132, wherein said processor is further operative to:
transition from the graphical representation to a view of detailed attributes associated with a database entry in response to a user initiated command.

155. The system of claim 132, wherein said processor is further operative to:
modify the graphical representation to display symbols representing database entries with specific attributes in accordance with a user determined set of display-filter attributes.

156. The system of claim 155, wherein the display-filter attributes filter symbols based on database entry attributes comprising:
species, molecular mass, cell cycle expression, developmental stage, taxonomic group, tissue specificity, subcellular location, cell type, experimental condition, molecular function of the database entry, cellular process which the entry is a part, source of the molecule from which it was purified, effect of the interaction on at least one interacting symbol, type of interaction.

157. The system of claim 156, wherein the type of interaction comprises:
database entries binding, inhibiting, or activating interaction.

158. A system for conducting complex searching of database entries, comprising:
a memory having program code stored therein;
a processor operatively connected to said memory for carrying out instructions in accordance with said stored program code, wherein said program code, when executed by said processor causes said processor to:
compile a set of predetermined search terms, wherein said search terms correspond to database entry attributes;
receive a user-created query in accordance with Boolean logical formulation;
save the user-created query in response to a user-initiated save search command;
search the database entries in accordance with a complex searching protocol, wherein the predetermined search terms are related to the fields of cellular, biological or physiological scientific research.

159. The system of claim 158, wherein said processor is further operative to:
modify the saved search wherein, the user combines the saved search with previously saved search.

160. The system of claim 158, wherein said processor is further operative to:
modify the saved search wherein, the user deletes a search term from the saved search.

161. The system of claim 158, wherein said processor is further operative to:
modify the saved search wherein, the user adds a search term from the saved search.

162. The system of claim 158, wherein:
the database entry corresponds to relational database schema describing entries in the system, each of which has attributes that are searchable.

163. The system of claim 162, wherein said processor is further operative to:
search data attributes based on the relational database schema.

164. The system of claim 158, wherein said processor is further operative to:
create pull down menus, wherein each menu defines attributes of the database entries corresponding to proteins, genes, compounds, complexes of molecules, or interactions of molecular moieties.

165. The system of claim 164, wherein said pull down menus are configured to be searchable for standardized ontologies.

166. The system of claim 165, wherein said processor is further operative to:
remove extraneous material from said pull down menus in accordance with predetermined protocols related to a search parameter.

167. The system of claim 158, wherein said processor is further operative to:
associating the saved search with a specific user;
presenting a list of previous saved searches associated with said specific user in response to a user-issued command.

168. The system of claim 158, wherein said processor is further operative to:
reorder of said search results according to user manipulation of column header organization buttons.

169. The system of claim 89, wherein said processor is further operative to:
import regularly structured table data automatically, using an interface on a user node.

170. The system of claim 158, wherein said processor is further operative to:
import regularly structured table data automatically, using an interface on a user node.

171. The system of claim 169, further comprising:
a search facility configured to search the table data on the user node.

172. The system of claim 169, further comprising:
a tool to import and automatically upload the table data.

173. The system of claim 169, wherein a user can import new database tables altering a system's schema.

174. The system of claim 173, wherein tables from the system are selectable.

175. The system of claim 173, wherein a description of the new table field is added.

176. The system of claim 173, wherein the display order of the new data is specified for user output analysis pages.
